(19) United States
(12) Patent Application Publication
(10) Pub. No.: US 2014/0227686 A1
(43) Pub. Date: Aug. 14, 2014

(54) STABILIZED CHEMICAL DEHYDRATION OF BIOLOGICAL MATERIAL

(71) Applicant: GENTEGRA LLC, Pleasanton, CA (US)

(72) Inventors: Michael Saghbini, Poway, CA (US); Michael Hogan, Tucson, AZ (US); Chunnian Shi, San Diego, CA (US); Brian Dalby, Carlsbad, CA (US); David Wong, San Marcos, CA (US)

(73) Assignee: GENTEGRA LLC, Pleasanton, CA (US)

(21) Appl. No.: 14/254,478

(22) Filed: Apr. 16, 2014

Related U.S. Application Data
(63) Continuation of application No. 13/081,436, filed on Apr. 6, 2011, now abandoned.
(60) Provisional application No. 61/321,269, filed on Apr. 6, 2010.

(51) Int. Cl. C12Q 1/68

(52) U.S. Cl. CPC C12Q 1/6806
USPC 435/6.1

(57) ABSTRACT

The present invention provides compositions and methods that enable the stabilization and storage of samples by contacting a sample with an assembly of particles, and reducing the water activity level of the contacted sample. By reducing the water activity level of the sample, the assembly of particles minimizes the degradation of the sample. Stabilizers may or may not be added to the assembly of particles to further minimize the degradation of the sample. Subsequently to storage in the assembly of particles, the samples are recoverable by eluting the assembly of particles with a fluid solution. In one embodiment, the entire assembly of particles will dissolve into the solution. In another embodiment, only part of the assembly of particles will dissolve into the solution. The assembly of particles provides the advantage that while it is porous, it comprises non-porous particulate material.

### CROSS-REFERENCE TO RELATED APPLICATIONS

This application in a continuation application of U.S. Ser. No. 13/081,436 filed Apr. 6, 2011, which claims the benefit of U.S. Provisional Application No. 61/321,269 filed Apr. 6, 2010, which is incorporated herein by reference.

### FIELD OF THE INVENTION

This invention relates generally to a method for stabilizing biological samples. In particular the invention provides a method for stabilizing blood and blood components and other bodily fluids, bacterial, fungal, viral, animal and plant cell cultures in fluid suspension. The invention also provides a method for stabilizing tissue and organ samples.

### BACKGROUND OF THE INVENTION

Water is a major component contributing to instability of collected biological material. Such biological material tends to be complex in nature and often contains damaging entities such as nucleases, proteases, and other degrading and modifying enzymes and other chemicals that require an aqueous environment for activity. The damaging entities must be immediately inactivated following sample collection to maintain biological material integrity. Additionally, certain biological materials, such as RNA, can spontaneously hydrolyze in the absence of exogenous enzyme activity, due to direct or metal-catalyzed addition of free water.

Some level of nuclease inactivation can be achieved in the liquid state (patent 652864-RNA later). However, excess free water content can still cause hydrolysis. Dehydration has been used historically to achieve dry state stability. However, even active dehydration systems, using vacuum or forced air, take hours to achieve such stable state and require expensive equipment and thus are hard to implement at the site of sample collection. Additionally, the time needed to achieve dryness increases proportionately with an increase in sample size, thus contributing to further instability for large samples. Thus, there is a need for a scalable method for biological sample stabilization without refrigeration, via sample dehydration and the addition of stabilizers and inhibitors of degradation that can be performed in the seconds-to-minutes time frame, without the use of mechanical drying equipment.

The present invention provide instantaneous stabilization of a biological sample, by rapid complexion of free water in the sample and by diffusional addition of stabilizers to the sample, for transport and or archiving for subsequent analysis of their constituents components, and propagation of living entities, if stabilizing cultured cells.

### SUMMARY OF THE INVENTION

The present invention provides methods, products, and kits (having components described herein) for stabilizing biological samples, including solid tissues derived from humans, animals and plants, as well as biological fluids such as blood urine, saliva, sputum, nasal discharges, lavages, tissue homogenates, by completely covering the sample in a crystalline water-soluble compound and reducing the water activity level of the biological sample. The present invention also provides methods for stabilizing extracts and purifications from biological samples, including DNA, RNA, polypeptides, viral samples, cell extracts, antibodies, and cell cultures. The invention further provides methods for stabilizing biological samples in fluid suspension.

In one aspect, the invention may comprise an assembly of particles for stabilizing one or more biomolecules comprising: particulate material comprising particles and said one or more biomolecules, wherein said biomolecules are retained on an outer surface layer of said particles and wherein said biomolecules have a water activity level substantially less than 1. The above invention may further comprise an outer surface layer comprising one or more stabilizers. In some instances, one or more of the biomolecules may comprise a nucleic acid, a polypeptide, blood, serum, plasma, cell, tissue, sputum, mucus, cerebrospinal fluid, hair, urine, stool, semen, a metabolite, an antibody, a lipid, or a combination thereof. In other instances, one or more biomolecules are selected form the group consisting of, a bodily fluid, a tissue homogenate, a cell culture, a crude biological extract, a purified biologic, and any combination thereof. In yet other instances, one or more biomolecules are selected from the group consisting of, a plant extract, a microbial extract, an animal extract, and any combination thereof. In the above invention, the biomolecules do not comprise d-Lysergic Acid Diethylamide or polio virus. In the above invention, one or more biomolecules may have a higher resistance from degradation than a biomolecule not retained by said assembly. The above invention may further comprise one or more biomolecules in contact with a solid support, wherein said solid support is selected from the group consisting of a swab, a sponge or a paper. In some instances, least a portion of said biomolecules are recoverable from said assembly of the above invention.

In one aspect, the invention may comprise an assembly of particles comprising: particulate material comprising one or more stabilizers on at least an outer surface of said particulate material. The above invention may further comprise one or more stabilizers located only on said outer surface. In some instances, the above invention comprises an assembly of particles that absorbs liquid upon contact of said liquid with said assembly. The invention may further comprise an assembly of particles comprising a biomolecule that coexists as a thin chemically dehydrated surface film on the particulate material. In different embodiments of this invention, a stabilizer may be selected from the group consisting of: anti-microbial agent, anti-oxidant, apoptosis inhibitors, buffer, chaotrope chelating agent, denaturing agent, detergent, hydroxyl radical scavengers, hydroperoxide-removing entities, metal chelator, nuclease inhibitor, plasticizers, protease inhibitors, protein modification inhibitor, protein precipitants, protein stabilizers, reactive oxygen scavengers, and reducing agent and any combination thereof. In some instances, the stabilizer is an oxidation inhibitor, a pyruvate inhibitor, an enzymatic activity inhibitor or a combination thereof. In some embodiments of the invention, the particulate material is a crystalline compound. In other embodiments of the invention, the particulate material is selected from the group consisting of: a monosaccharide, a disaccharide, a polysaccharide, an organic salt, an inorganic salt, and any combination thereof. In the above invention, a random packing of said particulate material may leave at least 20%, 25%, 30%, 35%, 40% or more as interstitial space. The above invention may further comprise individual particles of said particulate material that are (i) no bigger than 10 mm in their longest dimension and (ii) no smaller than 0.1 mm in their shortest dimension. Additionally, the above invention may further comprise an assembly of particles that has (i) volume of at least 0.2 mL, at least 0.5 mL, at least 0.7 mL, or at least 1.0 mL, or (ii) at least one dimension that is at least 0.1, 0.2, 0.3, 0.4, or 0.5 cm in length.

In one aspect, the invention may comprise an assembly of particles comprising: particulate material, wherein each particle of said particulate material comprises: (A) a core having a contact angle greater than 50 degrees and (B) an outer surface having a contact angle less than 50 degrees. In some instances, the particles may have a spherical or rhomboidal shape. In some instances, a packing of said particulate material of the above invention leaves at least 10% as an interstitial space. The above invention may further comprise an outer surface selected from the group consisting of a carboxyl group, an amine group, an amide group, a hydroxyl group, a sulfhydryl group and any combination thereof. In some embodiments, the core of the above invention comprises plastics such as polyurethane, polyalkelene glycol, or polyethylene or polycarbonate or nylon. The above invention may further comprise an outer surface with one or more stabilizers. The stabilizers may be selected from the group consisting of: anti-microbial agent, anti-oxidant, apoptosis inhibitors, buffer, chaotrope, chelating agent, denaturing agent, detergent, hydroxyl radical scavengers, hydroperoxide-removing entities, metal chelator, nuclease inhibitor, plasticizers, protease inhibitors, protein modification inhibitor, protein precipitants, protein stabilizers, reactive oxygen scavengers, and reducing agent and any combination thereof. Additionally, the stabilizers may be oxidation inhibitors, pyruvate inhibitors, enzymatic activity inhibitors, or any combination thereof. In the above invention, the assembly of particles may comprise particulate material comprising microparticles with sugar moieties on their surfaces. The above invention may comprise an assembly comprising at least 100, 1,000, 10,000, 100,000, or 1,000,000 particles, or an assembly with a volume of at least 0.1 cc, 0.2, 0.5 cc, 1 cc, 5 cc, or 10 cc. In other instances, the above invention may comprise an assembly comprising magnetic particles. The above invention may further comprise an assembly of particulates that are an affinity resin selected from the group consisting of, a resin with affinity for nucleic acids, a resin with affinity for proteins, a resin with affinity for specific proteins, a resin with affinity for antibodies, and any combination thereof. In the above invention, a random packing of said particulate material may leave at least 20%, 25%, 30%, 35%, 40% or more as interstitial space.

In one aspect, the invention comprises a method for stabilizing and recovering a sample comprising: contacting said sample with an assembly of particles thereby capturing free liquid molecules from said sample; and rehydrating said sample by applying a controlled volume of a liquid hydrant to said assembly of particles thereby recovering at least a portion of said sample. The above invention may further comprise particles with a surface layer that is water soluble. Additionally, the particles in the above invention may comprise a monosaccharide, a disaccharide, a polysaccharide, an organic salt, an inorganic salt, or any combination thereof. In some instances of the above invention, the contacting step results in solvation of a surface layer of said particles, wherein said surface layer has a thickness of at least 1, 2, 5, 10, 20, 50 or 100 microns. In some instances a controlled volume of said liquid hydrant is less than two times the volume of said assembly of particles. The above invention may further comprise a method for analyzing the stabilized sample. In the above invention, the volume of the assembly of particles may be at least 10%, 20%, 30%, 40%, 50%, 60%, 70%, 80%, 90%, 100%, 110%, 120%, 130%, 140%, 150%, 160%, 170%, 180%, 190%, 200%, 300%, 400%, 500%, 600%, 700%, 800%, 900%, or 1000% larger than the volume of said fluid. iN some embodiments of the above invention the contacting step results in solvation of a surface layer of said particles, wherein said surface layer has a thickness of less than 100 microns, less than 20 microns, or less than 10 microns. The above invention may further comprise a method, wherein said contacting step results in solvation of a surface layer of said particles and wherein said surface layer has a volume that is less than ⅓ of the volume of the assembly of particles. In other embodiments of the above invention, the method may further comprise adding one or more stabilizers to said hydrated sample prior to said contacting step. In some embodiments, the particles comprise one or more stabilizers in a surface region. In other embodiments, the assembly of particles may have a volume greater than said fluid. The above invention may further comprise a method where the particles comprise an insoluble and/or hydrophobic core and a soluble and/or hydrophilic surface. In the above invention, the method may comprise assembly of particles completely dissolves into solution upon rehydration of said stabilized sample. The above invention may further comprise an assembly of particles that only partially dissolve into solution upon rehydration of said stabilized sample. In other embodiments of the above invention only a surface layer of the assembly of particles dissolves into solution upon rehydration of said stabilized sample. The above invention may further comprise a method for air-drying the sample and assembly of particles after said contacting step. In the above invention the sample may comprise a DNA or a protein. In some instances, the sample is a biological sample carried by a solid support, wherein said solid support is a cotton swab, a filter paper, or a sponge. In other instances, the sample is a solid tissue or carried by a solid tissue. In yet other instances, the sample is a biological fluid sample. In some instances of the above invention, the method does not involve vortexing.

In one aspect, the invention comprises a method for making particles for sample storage comprising: applying one or more stabilizers to a particle, thereby adsorbing said stabilizers on at least an outer surface of said particle. In some instances the outer surface of the above invention is water soluble. The above invention may further comprise a method in which stabilizers are water soluble. In some instances, the stabilizers comprise monosaccharides, disaccharides, polysaccharides, an organic salt, an inorganic salt, urea, polyolefin, or a combination thereof. The invention may further comprise a method in which said applying is to a plurality of particles arranged in a matrix.

In one aspect, the invention comprises a method for making particles for sample storage comprising: modifying an outer surface of one or more particles having a contact angle greater than 50 degrees to form a modified outer surface having a contact angle less than 50 degrees. The above invention may further comprise modifying occurs by an amination or carboxylation step. In some instances, the invention further comprises the method of applying one or more stabilizers to said outer surface. In some instances the stabilizers may comprise monosaccharides, disaccharides, polysaccharides, an organic salt, an inorganic salt, urea, polyolefin, or a combination thereof.

In one aspect, the invention comprises a solution comprising: spheres comprising: (A) a core having a contact angle greater than 50 degrees, and (B) an outer surface having a contact angle less than 50 degrees, optionally sugar or other dissolvable material, optionally stabilizer(s), biomolecule(s), and a rehydrating solution. In some instances of the above invention the polymer comprises polyurethane, polyalkelene glycol, or polyethylene. In some instances of the above invention the biological sample is either a tissue sample or comprises a blood component. The above invention may further comprise an assembly of particles comprising a biomolecule that coexists in a chemically dehydrated state with the excess of said particulate material.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a method for stabilizing biological fluid, solid tissue and a swapped biological sample in the presence of one or more water soluble crystalline compounds with or without stabilizers, in accordance with an embodiment of the invention.

FIG. 2 illustrates “drying” of a fluid biomolecule sample, in accordance with an embodiment of the invention.

FIG. 3 illustrates the interstitial space of assemblies composed of spheres and rhomboids, in accordance with an aspect of an embodiment of the invention.

FIG. 4 provides an image of a polyethylene bead, in accordance with an aspect of an embodiment of the invention.

FIG. 5 shows results from recovery of saliva samples applied to excess sucrose and air dried overnight at ambient temperature, in accordance with an embodiment of the invention.

FIG. 6 shows recovery results from whole blood storage on an assembly of Sucrose recovery results of raw blood stored dry on an assembly of particles for 30 days, at RT, 45 C, 56 C.

FIG. 7 shows results from raw buffy coat stored dry on an assembly of particles, at RT, 56 C, 76 C with a variety of stabilizing formulations.

### DETAILED DESCRIPTION OF THE PREFERRED EMBODIMENTS

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

There are various problems associated with current methods and systems for storage of biomolecules. For example, filter paper technology remains a world-wide standard for dry-state, ambient temperature biomolecule preservation in forensics and medical microbiology, yet the inherent porous nature of filter paper makes recovery of the preserved sample difficult. As another difficulty, the two-dimensional nature of filter paper provides only a limited storage capacity for biomolecule samples. Consequently, those skilled in the art have attempted to improve the capacity of filter paper; however, such configurations have further compounded the first problem, the difficulty in recovering biomolecule samples from porous material, by increasing exposure of the biomolecule sample to additional porous material. In many instances, specialized chemistries are necessary for the recovery of the biomolecule sample from the filter paper storage systems, which increase the difficulty in field collection.

The present invention provides compositions and methods that enable the stabilization and storage of samples by contacting a sample with an assembly of particles, as discussed herein, and reducing the water activity level of the contacted sample. By reducing the water activity level of the sample, the assembly of particles minimizes the degradation of the sample. Stabilizers may or may not be added to the sample or to the assembly of particles to further minimize the degradation of the sample. Subsequently to storage in the assembly of particles, the samples are recoverable by eluting the assembly of particles with a fluid solution. In one embodiment, the entire assembly of particles will dissolve into the solution. In another embodiment, only part of the assembly of particles will dissolve into the solution. The assembly of particles provides the advantage that while it is porous, it comprises non-porous particulate material. Also, when the particles are non-soluble or poorly water soluble, the sample can be rehydrated in solution and separated from the particles using a pipette having a bore size smaller than the diameter of the particles. Thus, the assembly has the additional improvement over filter paper in that it provides greater storage area.

In one embodiment, the invention stabilizes a sample by completely covering it in an excess of an assembly of water-soluble particles that comes in direct contact with the sample. The available water content of the sample is rapidly adsorbed onto the surfaces of the assembly of particles. The adsorbed water dissolves a small fraction of the assembly of particles saturating any water complement remaining with the sample. Chemical dehydration is obtained by tying up the water content of a sample with the assembly of particles. This rapid reduction of water activity leads to stabilization of the sample. As a result of chemical dehydration, the previously-hydrated sample is retained with an excess of un-dissolved particle fraction. This preferable comprises the majority of the particle fraction.

Thus the present invention contemplates an assembly of particles for stabilizing one or more biomolecules comprising: particulate material and a biomolecule, wherein said biomolecules are retained on an outer surface layer of said particles and wherein said biomolecules have a water activity level substantially less than 1, or less than 1, less than 0.9, less than 0.8, less than 0.7, less than 0.6, less than 0.5, less than 0.4, less than 0.3, less than 0.2, less than 0.1 or less than 0.05.

The term “non-porous,” when used in reference to the assembly of particles, refers to an assembly where at least some of the particles are non-porous. However, the assembly itself may be porous as there are interstitial voids between the particles.

Non-porous, when used in reference to the individual particles, refers to particles that have the innate characteristic that such particles display a void volume VV which is less than about 1/10th that of the total volume VT of the material. Examples of non-porous particulate materials include, but are not limited to, ceramics (e.g., carbonnitrides, silicon-carbides, etc.), glass, glass fiber, nylon, polyvinyl chloride, polybutylene, polypropylene, polyethylene, 5 polycarbonate, polysaccharides, and monosaccharides. One aspect of this characteristic is that it enables recovery of samples from an assembly of particles through washing with a fluid solution.

As used herein, “assembly of particles” may be used interchangeably with the terms: “assembly” and “matrix.” The “assembly of particles” is capable of retaining the fluid content of a sample either by adsorbing, absorbing or a combination thereof the fluid content of a sample.

In one embodiment, the assembly of particles is a pure substance. In another embodiment the assembly of particles is a mixture of substances. In a preferred embodiment, the assembly of particles readily adsorbs water at its solid surface. In a more preferred embodiment, the assembly of particles readily adsorbs liquid water but is not hygroscopic. In one embodiment, the assembly of particles is “glued” together, like sucrose granules which form an ordinary sugar cube, to form a solid, porous granular structure. In another embodiment, the assembly of particles is a powder. The assembly of particles can take on various formations. It can form an aggregate. The particles can be randomly packed or packed in an ordered form or with a repeating pattern. In some instances, the packing of the particles is such of a closely packed hexagonal array such as described in U.S. Pat. No. 6,406,848. The assembly can be held in place in a vial or other container or it may be freestanding.

In a preferred embodiment, the assembly of particles is granular, where granular implies that the individual particles are non-porous and have a diameter or longest dimension greater than 0.1, 0.2, 0.5, 1, 2, or 5 mm In any of the instances herein, the particles have a diameter or longest dimension in the range of from 0.1 mm to 2 mm, 0.1 mm to 1.5 mm, or 0.1 mm to 1 mm In any of the instances herein, the particles can a diameter or longest dimension no greater than 5, 4, 3, 2, 1, 0.9, 0.8, 0.7, 0.6, 0.5, 0.4, 0.3, 0.2, or 0.1 mm.

In other instances, a particle of the invention has its shortest dimension no longer than 5 mm, 2, mm, 1 mm, 100 micron, 50 microns, 10 microns. In some instances, the shortest dimension is at least 10, 20, 50, 100, 120, 150, 200, 220, 250, 300, 320, 350, 300, 420, 450, 500, 520, 550, 600, 620, 650, 700, 720, 750, 800, 820, 850, 900, 920, 950, or 1000 microns. In addition, the shortest dimension of a particle of the invention can be 1-100 microns, 5-50 microns, or 10-30 microns.

In yet other instances, the assembly of particles overall has one dimension that is at least 0.1, 0.2, 0.3, 0.4, 0.5 cm.

In one example, an assembly or particles comprise spherical particles having an average diameter of about 500 microns. In such an embodiment, the spherical particles may be contacted with a sample comprising fluid having a volume that is up to 80%, 75%, 70%, 65%, 60%, 55%, 50%, 45%, 40%, 35%, 30%, 25%, 20%, 15%, 10%, 5%, 1%, 0.5%, or 0.1% of the total volume of the particles in the assembly. In some instances, the volume of fluid to be dehydrated is at least 0.1%, 0.5%, 1%, 5%, 10%, 15%, 20%, 25%, 30%, 35%, 40%, 45%, 50%, 60% or 65% of the volume of the particles in the assembly. Ideally, the fluid volume of sample fluid is captured or adsorbed by the outer layer of the particles.

When contemplating particles having different material in the core and the outer layer the ratio of volume of outer layer to volume of core (excluding outer layer) would be up to 80%, 75%, 70%, 65%, 60%, 55%, 50%, 45%, 40%, 35%, 30%, 25%, 20%, 15%, 10%, 5%, 1%, 0.5%, or 0.1%. In some examples, the volume of the outer layer to the volume of core (excluding outer layer) may be at least 0.1%, 0.5%, 1%, 5%, 10%, 15%, 20%, 25%, 30%, 35%, 40%, 45%, 50%, 60% or 65%. Any of the ranges herein can be used in combination with other ranges. However, even when particles are made of a homogenous material (such as sugar particles), the above ratio can be used to determine the amount of liquid that can be absorbed and dehydrated by the assembly of particles.

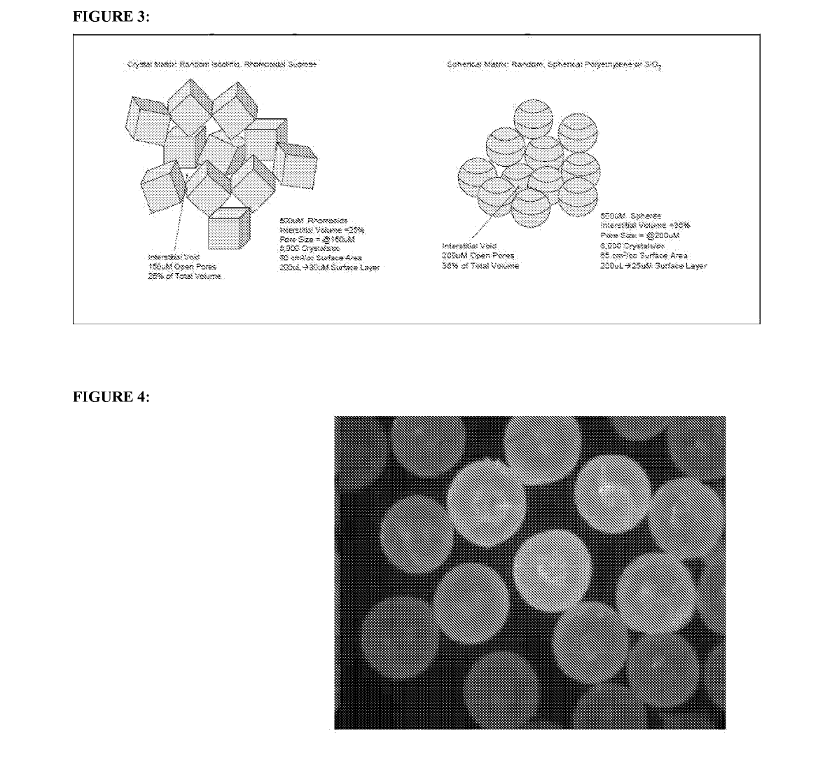

In the present invention, the individual particles of the assembly of particles may have a rhomboidal or a spherical shape, as indicated in FIG. 3.

In some embodiments, a packing of the individual particles of the assembly of particles will lead to an interstitial space between 10-15%, 15-20%, 20-25%, 25-30%, 30-35%, 35-40%, 40-45%, 50-55%, or in excess of 55% of total volume of the assembly. In other embodiments, a packing of the individual particles can lead to an assembly of particles with an interstitial space greater than 10%, 15%, 20%, 25%, 30%, 35%, 40%, 50%, or 55% of total volume of the assembly.

The assembly of particles may in some embodiments comprise at least 100, 1,000, 10,000, 100,000, or 1,000,000 particles. In other embodiments, the assembly may have a volume of at least 0.1 cc, 0.2, 0.5 cc, 1 cc, 5 cc, or 10 cc.

In one embodiment, the assembly of particles is selected from the group consisting of monosaccharide, disaccharide or polysaccharide. In a preferred embodiment, the assembly of particles is selected from the group consisting of sucrose, trehalose, maltose, fructose, mannitol, galactose, mannose, and combinations thereof. In one embodiment, assembly of particles is urea. In another embodiment, the assembly of particles is an organic salt such as sodium citrate or sodium oxalate or an inorganic salt such as sodium borate, ammonium sulfate, ammonium chloride or sodium chloride. Preferably the assembly of particles comprises or consists essentially of sugar or sucrose particles.

The present invention is not limited to the above listed compounds. Any water soluble assembly of particles can be used if it can induce chemical dehydration as described in the present invention.

In another embodiment, the assembly of particles comprises particles that have a core that is poorly water soluble. In some instances, the core of the particles comprises a plastic material such as, for example, polyurethane, polyalkelene glycol, polypropylene, nylon, or polyethylene. Thus the core of the particles may be completely or only partially insoluble in water. This provides the advantage that the core will not dilute the sample upon rehydration.

The particles herein can be further characterized as having a core having a contact angle greater than 50, 60, 70, 80, 90, or 100 degrees.

Such poorly soluble cores can have their surfaces modified, e.g., by aminiation or carboylation. In addition to, or in the alternative, such cores can have a surface layer added to them which is water soluble. The hydrophilic surface layer may be partially or completely soluble, specific non-limiting examples of hydrophilic surface layers include selections from the group consisting of a carboxyl group, an amine group, an amide group, a hydroxyl group, a sulfhydryl group and any combination thereof. Other examples of hydrophilic surfaces include the various saccharides described herein.

Preferably, the particles of the assemblies herein are characterized by having an outer surface with a contact angle of wetting for water between 0-40 degrees, 0-35 degrees, 0-30 degrees, 0-25 degrees, 0-20 degrees, 0-15 degrees, or less than 50, 40, 35, 30, 25, 20, 15, or 10 degrees.

A biological fluid may be applied directly to the assembly of particles, as illustrated in FIG. 1.1. In another embodiment, as illustrated in FIG. 1.2, a solid sample may be applied directly to the assembly. In yet another embodiment, illustrated in FIG. 1.3, a biological fluid or liquefied biological tissue is first transferred to a solid medium such as a swab, sponge or paper, which is then immediately placed into physical contact with an assembly of particles or crystalline compound, in the presence or absence of additional stabilizers and inhibitors of degradation, to stabilize the hydrated biological material associated with the solid medium. In the embodiments illustrated in FIGS. 1.2 and 1.3, the sample comprises a solid sample, which may additionally comprise free water molecules. In some embodiments, the solid medium is water soluble. In other embodiments, the solid medium itself is impregnated with stabilizers and inhibitors of degradation prior to biological fluid application. In some instances, the sample is washed away from the solid support (e.g., swab or sponge) onto the assembly of particles or crystalline compound of the present invention.

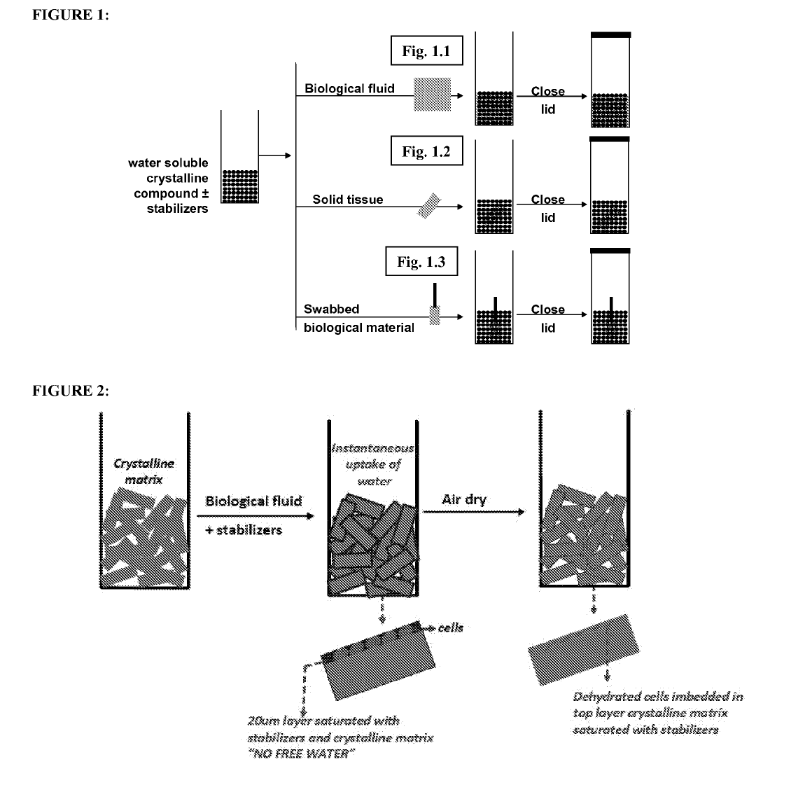

In one embodiment, illustrated in FIG. 2, biological fluid, according to the present invention, is spread thin, onto the surfaces of the assembly such that only part of the assembly solubilizes, thus immobilizing the biological fluid in a solid state impregnated with the non solubilized crystalline compound. All the water content of the biological fluid is tied up with the assembly of particles. In one embodiment, more of the particulate material is used than is necessary to immobilize the biological fluid. In another embodiment, only enough particulate material is used to immobilize the biological fluid. In a preferred embodiment, the amount of particulate material needed to completely immobilize a biological fluid is appropriately adjusted to fit the biological fluid volume. In another preferred embodiment, the particulate material comprises stabilizers and inhibitors of degradation that can quickly permeate a biological fluid for an added level of stability.

Biological tissue, according to the present invention, is contacted with an excess of the assembly of particles such that rapid transfer of tissue water onto the surface of the assembly of particles is achieved. Part of the assembly which comes in contact with the water content of the biological tissue is solubilized, thus diffusing into the tissue, to saturate and tie-up the remaining free water content of the biological tissue. In a preferred embodiment, the assembly comprises stabilizers and inhibitors of degradation that are quickly delivered into the tissue as part of the chemical dehydration process. In another preferred embodiment, the biological tissue is cut into thin pieces to allow rapid transfer and saturation of the deepest water in such tissue.

The sample, stabilized according to the present invention, may be allowed to go to dryness by exposing to ambient or heated air, or by drying in a vacuum system with or without heat. In some instances, the sample, stabilized according to the present invention, is not air dried, and may be immediately inserted into a vessel. In other embodiments, a drying cartridge is inserted into the vessel comprising the stabilized sample, thus allowing dehydration to occur in a closed system. In one embodiment, the sample, stabilized according to the present invention is stored at room temperature. In another embodiment, the sample, stabilized according to the present invention is stored at about 2 to about 8° C. In yet another embodiment, the sample, stabilized according to the present invention is stored at ambient temperature, or at −20° C., or at 4° C., or at 4-10° C., or at 10-20° C., or at 20-30° C. In other embodiments, the sample, stabilized according to the present invention is stored at a temperature greater than −20° C., 4° C., 10° C., 20° C., or 30° C.

In certain instances, an added level of stability can be obtained by incorporating stabilizers and inhibitors of degradation into the assembly of particles. Such stabilizers and inhibitors of degradation may be solubilized, completely or partially, by the water complement of the sample to quickly permeate into the sample. In one embodiment, stabilizers and inhibitors of degradation are added to the assembly of particles in a solid state format. In a preferred embodiment stabilizers and inhibitors of degradation are added in a liquid state and allowed to dry upon the surfaces of the assembly of particles prior to addition of biological material.

In some embodiments, stabilizers may be an intrinsic attribute of the assembly of particles, while in other instances, the assembly of particles may be modified with stabilizers. In some instances, the stabilizers may attach to the surface of the particulate material, or be embedded within the particulate material. In other instances, the stabilizers may be found alongside the particulate material within the assembly of particles. In yet other instances, the stabilizers are first added to the sample and then added to the assembly of particles. Stabilizers may be added to all types of samples including both fluid and solid samples.

Stabilizers may be selected from a variety of different compounds.

In some instances, a stabilizer is a material that is water soluble. For example, a stabilizer can be selected from the group consisting of monosaccharide, disaccharide or polysaccharide. In some cases, a stabilizer is selected from the group consisting of sucrose, trehalose, maltose, fructose, mannitol, galactose, mannose, and combinations thereof. A stabilizer can also be urea. The stabilizer can also be an organic salt such as sodium citrate or sodium oxalate or an inorganic salt such as sodium borate, ammonium sulfate, ammonium chloride or sodium chloride. In some instances, a stabilizer is not a sugar. In some instances, the stabilizer is not a salt. In some instances, a stabilizer is not urea.

In preferred embodiments, stabilizers slow the degradation of a sample stored the particles. Stabilizers may be selected from the group consisting of: anti-microbial agent, anti-oxidant, apoptosis inhibitors, buffer, chaotrope, chelating agent, denaturing agent, detergent, hydroxyl radical scavengers, hydroperoxide-removing entities, metal chelator, nuclease inhibitor, plasticizers, protease inhibitors, protein modification inhibitor, protein precipitants, protein stabilizers, reactive oxygen scavengers, reducing agents, inhibitors of other degrading and modifying enzymes, albumin, casein, collagen, pH stabilizers, and combinations thereof.

In more particular aspects, pH stabilizers may include those selected from potassium chloride, citric acid, potassium hydrogenphthalate, boric acid, potassium dihydrogenphosphate, Diethanolamine, sodium citrate, sodium dihydrogenphosphate, 30 sodium acetate, sodium carbonate, sodium tetraborate, cacodylic acid, imidazole, and 2-Amino-2-methyl-1-propanediol. In more particular aspects, the chelating agent is optionally selected from EDTA (Ethylenediamine-tetraacetic acid), EGTA (Ethyleneglycol-0,0′-bis(2-aminoethyl)-N,N,35N′,N′-tetraacetic acid), GEDTA (Glycoletherdiaminetetraacetic acid), HEDTA (N-(2-Hydroxyethyl)ethylenediamine-N,N′,N′-triacetic acid), NTA (Nitrilotriacetic acid), Salicylic acid and Triethanolamine In more particular aspects, the denaturing agent or detergent is an anionic surfactant, nonionic surfactant, cationic surfactant or ampholytic surfactant, which is optionally selected from SDS, Sodium lauryl sulfate, NP40, triton X-100, Sodium cholate, Sodium deoxycholate, Benzethonium chloride, CTAB (Cetyltrimethylammonium bromide), Hexadecyltrimethylammonium bromide and N,N-Dimethyldecylamine-N-oxide. In more particular aspects, the reducing agent or antioxidant is a free radical scavenging agent, or is optionally selected from DTT (dithiothreitol), dithioerythritol, urea, uric acid, mercaptoethanol, dysteine, vitamin E, vitamin C, dithionite, thioglycolic acid and pyrosulfite. In more particular aspects, the protease inhibitor is a serine or cysteine protease inhibitor, and is optionally selected from PMSF, PMSF Plus, APMSF, antithrombin III, Amastatin, Antipain, aprotinin, Bestatin, Benzamidine, Chymostatin, calpain inhibitor I and II, E-64, 3,4-55 dichloroisocoumarin, DFP, Elastatinal, Leupeptin, Pepstatin, 1,10-Phenanthroline, Phosphoramidon, TIMP-2, TLCK, TPCK, trypsin inhibitor (soybean or chicken egg white), hirustasin, alpha-2-macroglobulin, 4-(2-aminoethyl)-benzenesulfonyl fluoride hydrochloride (AEBSF) and a Kunitz-type protease inhibitor. In more particular aspects, the antimicrobial is an anti-biotic, anti-viral, anti-fungal or antiparasitic agent; is a member of a class selected from: betalactams; semisynthetic penicillins; monobactams; carboxypenems; aminoglycosides; glycopeptides; glucan synthesis inhibitors; Lincomycins; macrolides; polypeptides; allylamines; azoles; polyenes; sulfonamides; pyrimidines; tetraenes; thiocarbamates; benzoic acid compounds, complexes and derivatives thereof; rifamycins, tetracyclines, reverse transcriptase inhibitors, protease inhibitors, thymidine kinase inhibitors, sugar or glycoprotein synthesis inhibitors, structural protein synthesis inhibitors, nucleoside analogues, and viral maturation inhibitors, or is optionally selected from: penicillin, cephalosporin, ampicillin, amoxycillin, aztreonam, clavulanic acid, imipenem, streptomycin, gentamycin, vancomycin, clindamycin, polymyxin, erythromycin, bacitracin, amphotericin, nystatin, rifampicin, tetracycline, chlortetracycline, doxycycline, chloramphenicol, ammolfine, butenafine, naftifine, terbinafine, ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, undecylenate, silver sulfadiazine, undecylenic acid, undecylenic alkanolamide, Carbol-Fuchsin, nevirapine, delavirdine, efavirenz, saquinavir, ritonavir, indinavir, nelfinavir, amprenavir, zidovudine (AZT), stavudine (d4T), lamivudine (3TC), didanosine (DDI), zalcitabine (ddC), abacavir, acyclovir, penciclovir, valacyclovir and ganciclovir.

The present invention, may in certain embodiments also comprise particulates that are an affinity resin selected from the group consisting of, a resin with affinity for nucleic acids, a resin with affinity for proteins, a resin with affinity for specific proteins, a resin with affinity for antibodies, and any combination thereof.

A sample, according to the present invention, may include a solid or liquid sample. Additionally, a sample may include a biomolecule, biological sample, specimen or any combination thereof. In some instances, a sample may be selected from the group consisting of a bodily fluid, a tissue homogenate, a cell culture, a crude biological extract, (such as, a plant extract, a microbial extract, an animal extract, and any combination thereof) a purified biologic, or solid tissues derived from humans, animals and plants, blood, serum, plasma, biopsied cells or tissues, sputum, mucus, cerebrospinal fluid, hair, urine, stool, semen, nasal discharge, urine, lavages, saliva tissue homogenates and any combination thereof. In other instances, a sample may comprise a member from the group of nucleic acid, polypeptide, metabolites, antibodies, lipids and any combination thereof. In yet other instances, a sample may comprise any compound that would benefit from dry-state storage. While particular embodiments of samples are shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention.

As used herein, the term biomolecule may refer to any molecule typically found or produced by a living or non-living organism, or a sample containing such a material. Biomolecules therefore include organic molecules, such as peptides (protein), nucleic acid (polynucleotides), carbohydrates, sugars, fatty acids, lipids, as well as combinations thereof and in combination with inorganic molecules. Typically, a sample present or produced by a living or non-living organism includes a plurality of such biomolecules. A biomolecule can therefore be a part of a larger sample, which can include one or more peptide, nucleic acid, carbohydrate, sugar, fatty acid and lipid alone or in any combination. Thus, a peptide or nucleic acid retained by an assembly of particles may or may not include one or more additional biomolecules absorbed to the assembly. Consequently, a given biomolecule absorbed to the assembly may be alone or in a combination with one or more additional biomolecules absorbed to the assembly.

Biomolecules can be obtained, isolated or derived from, inter alia, living or non-living organisms, or anything produced by living or non-living organisms. Specific non-limiting examples include mammalian animals (e.g., primates including humans, apes, chimpanzees, gibbons; and farm and domestic animals including canine, feline, bovine, equine and porcine), which are typically warm-blooded, and non-mammalian animals (e.g., reptilian and avian), which are typically cold-blooded. Biomolecules can be isolated or obtained from tissues, organs, cells. Biomolecules can be isolated or obtained from microorganisms, including, for example, bacteria, fungi, parasites, virus and mycoplasma.

Biomolecules can include mixtures of cells (e.g., a tissue or organ biopsy), a particular cell type (e.g., hematopoetic cells), or a part of a cell, such as a protein or nucleic acid extract from a mixture of cells or particular cell type. The biomolecule can therefore be from or derived from any kind of cell, including prokaryotic and eukaryotic cells. An assembly may therefore have absorbed thereto any type of prokaryotic or eukaryotic cell, a part of a cell, and may include a mixture or collection of cells.

Cells include unicellular eukaryotes, multicellular eukaryotes, or a sample of cells (e.g, a tissue or organ sample or biopsy) from a multicellular eukaryote. The eukaryotic cell can be, for example, a blood cell or a tissue cell. Prokaryotic cells include eubacteria and archaebacteria, and gram-positive and gram-negative bacteria. The prokaryote can be a pathogenic or non-pathogenic organism. Biomolecules include a sample or material from a single or individual organism (e.g., a human subject), a single species (e.g., a subpopulation of human subjects), a plurality of organisms, or a plurality of species.

Biomolecules include a specimen also referred to as material, obtained from an organism. Biomolecules include a specimen obtained from a subject. Biomolecules include tissue, blood, serum, plasma, cerebral spinal fluid, hair, fur, saliva, sputum, semen, urine, stool, mucous, skin, a benign or malignant tumor or growth, biopsied organ, tissue or any other type of cell, organ or tissue sample or material, optionally in solution or in suspension.

Biomolecules can be derived or obtained from a plant or plant part, for example, leaf, stem, stalk, pollen, root, branch, flower, seed, bulb, spore or other plant material. Biomolecules are present in food, forensic samples, agricultural samples and products as well as environmental samples (e.g., soil, dirt, fresh water, salt water or waste water, landfill material, garbage or waste). Biomolecules can also be artificial or synthetically produced. For example, synthetic methods of producing peptides, nucleic acids, fats, lipids, carbohydrates are known in the art.

In the present invention, recovery of samples stabilized by the current invention may be achieved by re-hydration of the sample along with partial or complete hydration of the assembly, by adding liquid hydrant, or a buffered solution, or an osmotically balanced solution, or growth media, if propagation is desired after rehydration. In the case of a solid tissue, excess particles can be removed prior to tissue processing.

The volume of liquid sufficient to adequately hydrate the assembly of particles to recover a sample absorbed to the assembly may vary depending on the particulate material composing the assembly. In some embodiments, the assembly may comprise particulate material that is entirely soluble. In such embodiments, recovery of samples may require hydration with a volume of solution equivalent to, or in excess of, the volume of the assembly. In other embodiments, the assembly may comprise particulate material that is only partially soluble. In such embodiments, recovery of sample may require hydration with a volume of solution equivalent to, or in excess of, the volume of the soluble portion of the assembly of particles. One advantage of a partially soluble assembly of particles is that recovery of sample may not require as much fluid to rehydrate the sample, thus minimizing the dilution. Additionally, a partially soluble assembly provides the advantage that only a portion of the assembly will dissolve into the solution upon rehydration of sample, thus minimizing interference of the soluble assembly, if any, with downstream processing and analysis.

The assembly may be such that applying a fluid to an assembly of particles comprising one or more biomolecules (e.g., peptide or nucleic acid) absorbed thereto elutes or recovers at least a portion of the biomolecule from the assembly. In particular aspects, 30-50%, 50-65%, 65-80%, 80-90%, or more of the biomolecule (e.g., peptide or nucleic acid) is recovered from an assembly upon applying a fluid (e.g., an aqueous liquid such as water) to the assembly. In more particular aspects, the aqueous liquid has a pH within a range of 5.0 to 9.0, has a pH within a range of 10 to 12, 11 to 12, 11.3 to 11.8, 11.4 to 11.7, or a pH of about 11.4, 11.5, 11.6, 11.7, or 11.8, or has a stabilized pH. In further particular aspects, stabilization of pH can be achieved with a zwitterion, with Tris (hydroxymethyl)aminomethane hydrochloride (TRIS), N-(2-hydroxyethyl)piperazine-N′-2-ethanesulfonic acid (HEPES), 3-(N-morpholino) propanesulfonic acid (MOPS), 2-(N-morpholino) ethanesulfonic acid (MES), N-tris[hydroxymethyl]methyl-2-aminoethanesulfonic acid (TES), N-[carboxymethyl]-2-aminoethanesulfonic acid (ACES), N-[2-acetamido]-2-iminodiacetic acid (ADA), N,N-bis[2-hydroxyethyl]-2-aminoethanesulfonic acid (BES), N-[2-hydroxyethyl]piperazine-N′-[2-hydroxypropoanesulfonic acid] (HEPPSO), N-tris [hydroxymethyl]methylglycine (TRICTNE), N,N-bis[2-hydroxyethyl]glycine (BICINE), 4-(cyclohexylamino)-1-butanesulfonic acid (CABS), 3-(cyclohexylamino)-1-propanesulfonic acid (CAPS), 3-(cyclohexylamino-2-hydroxy-1-propanesulfonic acid (CAPSO), 2-(cyclohexylamino) ethanesulfonic acid (CHES), N-(2-hydroxyethyl)piperazine-N′-(3-propanesulfonic acid) (EPPS), piperazine-N,N′-bis(2-ethanesulfonic acid (PIPES), [(2-hydroxy-1,1-bis[hydroxymethyl]ethyl)amino]-1-propanesulfonic acid (TAPS), 2-amino-2-methyl-1-propanol (AMP), 5 3-[(1,1-dimethyl-2-hydroxyethyl)amino]-2-hydroxypropanesulfonic acid (AMPSO), ethanolamine, or 3-amino-lpropanesulfonic acid.

Samples including biomolecules, such as peptide or nucleic acid eluted or recovered from the assembly, can subsequently be used for any analytical, functional or structural analysis or application, if desired. For example, a biomolecule absorbed or adsorbed to an assembly can be analyzed in situ, wherein the biomolecule is analyzed without elution or recovery from the assembly. As an example, elution liquid added to peptide or nucleic acid absorbed to the assembly, and regents for subsequent analysis 65 (e.g. calorimetric reagents) are added to the same vessel housing the assembly. Thus, a subsequent analysis or application does not require elution or recovery of a biomolecule from the assembly, but if a biomolecule is eluted or recovered from the assembly, it will be in a form amenable to a subsequent analysis or application.

Non-limiting examples of subsequent analysis which may be performed on biomolecules include enrichment, purification, sequencing, molecular weight analysis, isoelectric point analysis, charge density analysis, structural analysis or crystallization. Additional examples of subsequent analysis include functional assays, such as binding affinity or enzymatic or catalytic activity. Additional examples, include electrophoresis, purification, sequencing, molecular weight analysis, structural analysis, functional assays, such as binding or hybridization. Additional examples of nucleic acid subsequent analysis include genotyping, fingerprinting, expression of recovered nucleic acid (transcription or translation), cloning or other genetic manipulation. Further examples of nucleic acid subsequent analysis include synthesis or amplification (e.g., polymerase chain reaction, PCR, ligase chain reaction, LCR, reverse transcriptase initiated PCR, rtPCR and whole genomic amplification via PCR-based or isothermal amplification methods), DNA or RNA hybridization techniques including restriction fragment length polymorphism, RFLP, sequencing, STR and SNP analysis, and applications to microarrays, gene chips, and any high-throughput or automated application, analysis or process.

Biomolecules can optionally be enriched or purified, and subjected to a subsequent analysis or application. For example, nucleic acid can be purified prior to cloning, amplification or other genetic manipulation. Biomolecules can also be subjected to labeling reactions, such as peptide or nucleic acid labeled with a radioisotope for use as a probe or a primer. More specifically, for example, nucleic acid or peptide recovered from a blood sample absorbed to an assembly may be sequenced or size fractionated on an agarose or polyacrylamide gel for purification, enrichment or for analysis.

In some embodiments, the assembly of particles may store either viruses or bacteria. In such embodiments, the viruses and bacteria may retain viability, or if desired, have reduced or no viability depending on the composition of the assembly of particles and on the type of surface coatings applied. For example, an acidic or basic coating may be added to the assembly of particles. A nonexclusive list of surface coatings for those may include: citrate or a weak base like Tris, detergents, anionic detergent like SDS, cationic detergents like CTAB, and non-ionic detergents like Tween-100 or NP-40.

In one embodiment, the particles of the invention are composed of a homogenous material, e.g., are sugar or sucrose particles. In another embodiment, the particles are salt particles (e.g, such as inorganic salt or organic salt).

In one embodiment the biological material is stabilized, by contact with the assembly of particles, with respect to its constituent components (i.e. nucleic acids, proteins, metabolites, lipids, etc.) individually or combined. In another embodiment, foreign pathogens present within a biological material, at the time of collection, are stabilized. In a preferred embodiment a biological culture comprising mammalian, bacterial, fungal, plant or vial cells is stabilized by contact with the assembly to retain viability such that upon subsequent re-hydration and transfer to appropriate growth conditions, cells are then able to propagate.

In one embodiment, the individual particles of the assembly comprise an insoluble core modified with a hydrophilic surface layer. The hydrophilic surface layer may be added to the insoluble core in a number of ways. In one embodiment, an amino surface is introduced using standard low-temperature vacuum amination, which can be performed directly upon the insoluble core. In other embodiments, carboxylate is similarly added to the insoluble cores. These simple low temperature gas-phase modifications may be used to confer wetting, hydrophilic characteristics to the insoluble cores with a variety of hydrophilic groups.

The present invention also provides methods for modifying an assembly of particles to include magnetic beads. In one embodiment, applying the magnetic beads as a suspension, along with other stabilizers, or alone, during the molding of the assembly of particles will achieve this goal.

In additional embodiments, a biomolecule (e.g., peptide or nucleic acid) adsorbed, absorbed or both to the assembly of particles resists degradation as compared to unabsorbed biomolecule (e.g., peptide or nucleic acid). In one aspect, peptide adsorbed to the assembly resists degradation as compared to unabsorbed peptide. In another aspect, nucleic acid adsorbed to the assembly resists degradation as compared to unabsorbed nucleic acid. In particular aspects, the resistance to degradation comprises a loss of no greater than 75%, 50%, 33%, 25%, 15%, 5%, or any range in between of the biomolecule (e.g., peptide or nucleic acid), as compared to an equivalent amount of unabsorbed biomolecule (e.g., peptide or nucleic acid), over a period of time; or the resistance to degradation comprises preserving greater than 33%, 50%, 75%, or 90% or more of the biomolecule (e.g., peptide or nucleic acid), as compared to an equivalent amount of unabsorbed biomolecule (e.g., peptide or nucleic acid), over a period of time, for example, for 5-10, 10-20, 20-30, 30-50, 50-90, 50-150, 150-365 days or weeks, or for 1, 2, 3, 4, 5, 6, 7, 8, 9, 10 years, or more (e.g., at ambient temperature, at −20° C., at 4° C., at 4-10° C., at 10-20° C., or at 20-30° C.). In the context of DNA, resistance to degradation may provide less than 1 DNA strand break per 10 K by per month, 6 months, or 1 year storage at ambient temperature.

Degradation can be assessed, for example, by determining one or more of the quantity of the biomolecule (e.g., peptide or nucleic acid) or a fragment of the biomolecule (e.g., peptide or nucleic acid); size fractionation and determining the relative amount of biomolecule (e.g., peptide or nucleic acid) or a fragment of the biomolecule (e.g., peptide or nucleic acid); by direct or indirect quantitation of biomolecule (e.g., peptide or nucleic acid) fragmentation; by measuring biological activity, if any, of biomolecule (e.g. peptide) or by the amount of phosphorylation or prenylation (e.g., peptide).

In one embodiment, the biological material, stabilized according to the present invention, is shipped at ambient temperature. In another embodiment, the biological material, stabilized according to the present invention, is shipped at −20° C., at 4° C., at 4-10° C., at 10-20° C., or at 20-30° C.

In one embodiment, the assembly is provided in a multi-sample container (i.e. a plate) which can be sealed after addition of biological material. In a preferred embodiment the assembly of particles is provided in an individual sealable container. In another preferred embodiment the assembly of particles is provided in sealed pouches, like a sugar packet, the content of which is added to the biological material once placed in a sealable container.

The assembly of particles shape will be determined, in part, by any housing (e.g., vessel or tube) or storage unit containing the assembly of particles. Exemplary sizes range from 1-5 mm3, 5-10 mm3, 10-20 mm3, 20-30 mm3, 30-50 mm3, 50-100 mm3, 100-200 mm3, 200-500 mm3, 500-1000 mm3, 1-5 cm3, 5-10 cm3, 40 10-20 cm3, 20-30 cm3, 30-50 cm3, 50-100 cm3, 100-200 cm3, 200-500 cm³, or more, or any numerical value or range within such ranges. An exemplary assembly of particles is a 5 mm high×6 mm wide cylinder, which has a volume of about 150 mm3 Exemplary non-limiting assembly of particles shapes include rectangular, square, cylindrical, circular, spherical and triangular.

The invention provides kits including invention compositions (e.g., “absorbed assembly units,” which as set forth herein, include, inter alia, a biomolecule such as a peptide or nucleic acid absorbed to an elutable assembly which is elutable or recoverable, at least in part, from the assembly). In one embodiment, a kit includes an absorbed assembly unit, which includes a peptide and an elutable assembly substantially free of moisture, wherein the peptide is absorbed to the assembly, wherein the peptide resists degradation as compared to unabsorbed peptide, and wherein at least a portion of the peptide is recoverable or elutable from the assembly, packaged into suitable packaging material. In another embodiment, a kit includes an absorbed assembly unit, which includes a nucleic acid absorbed to the assembly to which the peptide is absorbed. In a further embodiment, a kit includes an absorbed assembly unit, which includes a peptide, a nucleic acid and an elutable assembly substantially free of moisture, wherein the peptide and the nucleic acid is absorbed to the assembly, wherein the peptide or the nucleic acid resists degradation as compared to unabsorbed peptide or nucleic acid, and wherein at least a portion of the peptide or the nucleic acid is recoverable or elutable from the assembly.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention. Kits of the invention therefore can additionally include labels or instructions for using one or more of the kit components in a method of the invention. Instructions can include instructions for practicing any of the methods of the invention described herein. The instructions may be on “printed matter,” e.g., on paper or cardboard within the kit, or on a label affixed to the kit or packaging material, or attached to a vial or tube containing a component of the kit. Instructions may additionally be included on a computer readable medium, such as a disk (floppy diskette or hard disk), optical disk such as CD- or DVD-ROM/RAM, DVD, MP3, magnetic tape, or an electrical storage media such as RAM and ROM and hybrids of 20 these such as magnetic/optical storage media.

In some embodiments, kits may further include a plurality (two or more) of absorbed assembly units. In one aspect, each absorbed assembly unit includes a peptide and an elutable assembly substantially free of moisture, wherein the peptide is absorbed to the assembly, wherein the peptide resists degradation as compared to unabsorbed peptide, and wherein at least a portion of the peptide is recoverable or elutable from the elutable assembly. In another aspect, each absorbed assembly unit includes a peptide, a nucleic acid and an elutable assembly substantially free of moisture, wherein the peptide and the nucleic acid is absorbed to the assembly, wherein the peptide or the nucleic acid resists degradation as compared to unabsorbed peptide or nucleic acid, and wherein at least a portion of the peptide or the nucleic acid is recoverable or elutable from the elutable assembly.

An additional example of an invention kit includes a package having one or more compartments and an assembly of particles as described herein, each compartment having a physical size sufficient for holding an assembly, wherein the assembly comprises a material suitable for absorbing a biomolecule (e.g., peptide or nucleic acid) and for elution or recovery of the absorbed biomolecule from the elutable assembly; and, instructions for absorbing a biomolecule (e.g., peptide or nucleic acid) to the elutable assembly. Accordingly, invention kits include elutable assembly suitable for absorbing a biomolecule (e.g., peptide or nucleic acid) in which a biomolecule (e.g., peptide or nucleic acid) has not yet been absorbed to the elutable assembly present in the kit.

Kits of the invention may contain an elution or recovery liquid, an optional wash solution, and one or more other additional components useful for elution or recovery of biomolecules. Kits of the invention may contain an elution or recovery liquid, an optional wash solution, and one or more other additional components useful for analysis of the eluted or recovered nucleic acid. A kit may further include one or more reagents useful for amplifying a nucleic acid of interest, including but not limited to, one or more amplification primers, one or more dioxy nucleotide triphosphates (e.g., a mixture of dATP, dGTP dCTP and/or dUTP or dTTP) one or more polymerizing enzymes (e.g., DNA polymerase), etc. A kit may include one or more additional reagents useful for sequencing a nucleic acid of interest, for example, one or more sequencing primers (labeled or unlabeled, or covalently modified), one or more deoxynucleotidetriphosphates (e.g., a mixture of dATP, dGTP, dCTP and dUTP or dTTP), one or more labeled or unlabeled dideoxynucleotide triphosphate terminators (e.g., ddATP, ddGTP, ddCTP and ddUTP or ddTTP) or one or more polymerizing enzymes (e.g., DNA polymerase, Taq polymerase, Pfu, elongase). A kit may include one or more reagents useful for labeling an isolated nucleic acid, e.g., one or more labeled deoxynucleotide triphosphates, one or more polymerizing enzymes, or one or more labeled or unlabeled primers.

Individual absorbed assembly units can be included within a storage unit. A storage unit is a structure (container or housing) that can be used to house or store one or more (e.g., a plurality) assembly units. Thus, a storage unit can contain single or multiple compartments for elutable assemblies or absorbed assembly units. In one embodiment, the storage unit includes one or more absorbed assembly units in which peptide is absorbed to an elutable assembly, which is substantially free of moisture, wherein the peptide resists degradation as compared to unabsorbed peptide, and wherein at least a portion of the peptide is recoverable or elutable from the elutable assembly. In another embodiment, a storage unit includes one or more absorbed assembly units in which a nucleic acid is absorbed to an elutable assembly, which is substantially free of moisture, wherein the nucleic acid resists degradation as compared to unabsorbed nucleic acid, and wherein at least a portion of the nucleic acid is recoverable or elutable from the assembly. In yet another embodiment, a storage unit includes one or more absorbed assembly units in which a peptide and a nucleic acid are absorbed to an elutable assembly, which is substantially free of moisture, wherein the peptide or the nucleic acid resists degradation as compared to unabsorbed peptide or nucleic acid, and wherein at least a portion of the peptide or the nucleic acid is recoverable or elutable from the assembly. In particular aspects, a storage unit includes two or more absorbed assembly units (e.g., 3, 4, 5-10, 10-25, 25-50, 50-100, 100-500, 500-1000, 1000-5000, 5000-10,000, or any numerical value or range within such ranges), each of which have a different peptide or a different nucleic acid. In additional particular aspects, a storage unit includes two or more absorbed assembly units (e.g., 3, 4, 5-10, 10-25, 25-50, 50-100, 100-500, 500-1000, 1000-5000, 5000-10,000, or any numerical value or range within such ranges), each of which have a different biological sample.

Elutable assemblies can be included with a storage unit. In one embodiment, a storage unit has a plurality of compartments each having a physical size sufficient for housing an elutable assembly and one or more elutable assemblies, in which the elutable assembly is suitable for absorbing a biomolecule. Typically, the elutable assembly is a material suitable for storing or preserving a biomolecule (e.g., peptide or nucleic acid) and for elution or recovery of the biomolecule from the elutable assembly. Such storage units can also include instructions for absorbing a biomolecule (peptide or nucleic acid) to the elutable assembly, instructions for elution or recovery of the absorbed biomolecule from the elutable assembly, or instructions for preparing an aqueous liquid for eluting or recovering the absorbed biomolecule from the elutable assembly. Accordingly, invention storage units include units housing elutable assembly suitable for absorbing a biomolecule (e.g., peptide or nucleic acid), in which a biomolecule (e.g., peptide or nucleic acid) has not yet been absorbed to the elutable assembly present in the unit.

A kit or storage unit typically includes a label or packaging insert including a description of the components or instructions for use. Exemplary instructions include, instructions for eluting or recovering at least a portion of one or more biomolecules such as peptide or nucleic acid alone or in combination, either preferentially, sequentially or simultaneously; instructions for eluting or recovering at least a portion of a peptide alone or in combination with at least a portion of the nucleic acid, either preferentially, sequentially or simultaneously; or instructions for absorbing a biomolecule, such as peptide or nucleic acid or sample thereof, to an elutable assembly.

Additional optionally included or excluded components of invention kits and storage units include, for example, a liquid suitable for elution or recovery of a biomolecule absorbed to an assembly. In one aspect, the liquid is aqueous, and is suitable for elution or recovery of a peptide or a nucleic acid from an elutable assembly. In additional aspects, kits and storage units include liquid suitable for elution or for recovery preferentially, sequentially or simultaneously a biomolecule (e.g., peptide or nucleic acid) from an elutable assembly, or at least a portion of a biomolecule (e.g., peptide or nucleic acid) from an elutable assembly. In yet additional aspects, kits and storage units include instructions for preparing an aqueous liquid for eluting or recovering a biomolecule (e.g., peptide or nucleic acid) from one or more of the plurality of elutable assemblies.

A kit or storage unit can contain additional components, for example, a device (vessel or holder) having a physical size sufficient for holding an elutable assembly, and optionally suitable for eluting or recovering at least a portion of the peptide from an absorbed assembly unit, at least a portion of the nucleic acid, or at least a portion of the peptide in combination with at least a portion of the nucleic acid from the assembly unit. In one aspect, the device (vessel or holder) has a physical size sufficient for introducing or holding an elutable assembly, the device having an open end, an openable end or a removable end, and wherein the device (vessel or holder) has physical dimensions suitable for inserting a plunger therein so as to cause compression of the elutable assembly. In another particular aspect, the device (vessel or holder) has a physical size sufficient for introducing or holding an elutable assembly, in a physical configuration, such as a tube or spin column, suitable for insertion into a centrifuge tube. A plurality of such devices each having a physical size sufficient for introducing or holding one or more assembly units can also be included in a kit. A plurality of such devices (vessels or holders) is amenable to automated handling of multiple assembly units for elution or recovery of biomolecules from each assembly unit.

Kits may further include tools for manipulating elements for biomolecule elution or recovery, vessels or holders for collecting eluted or recovered biomolecules, materials for purifying biomolecules. For example, columns or cartridges for peptide or nucleic acid purification from a solution, affinity media such as beads for peptide or nucleic acid purification from a solution, or chromatographic media for purification or separation of peptide or nucleic acid can be included in a kit. Materials for subsequent purification of eluted nucleic acids include, but are not limited to, magnetic beads for nucleic acid purification, and nucleic acid purification columns.

Individual storage units (containers or housings) can comprise any physical configuration suitable for housing one or more elutable assemblies, including an absorbed assembly unit as set forth herein, having a stored or preserved biomolecule. Each of the absorbed assembly units can have a defined location, position or address within the storage unit. In one embodiment, a storage unit comprises a multi-well plate. In particular aspects, a multi-well plate comprises 2-6, 6-12, 12 to 24, 24-96, or more compartments. In additional particular aspects, one or more of the wells of the multi-well plate has a volume of about 10-50 ul, 50-100 ul, 100-250 ul, 250-500 ul, 0.5-1.0 ml, 1.0-2.0 ml, 2.0-3.0 ml, 3.0-5.0 ml, or 5.0-10.0 ml, more particularly, 50 ul, 100 ul, 200 ul, 250 ul, 500 ul, or any numerical value or range within such ranges.

Storage units also refer to a plurality of two or more individual storage units. Thus, as used herein a storage unit also refers to a plurality of individual apparatus or container for housing one or more elutable assemblies. In one embodiment, a storage unit houses a plurality of stored or preserved peptides, each peptide individually adsorbed to an elutable assembly substantially free of moisture, wherein at least a portion of said peptide is recoverable or elutable from said elutable assembly.

A storage apparatus can be used to house or store adsorbed assembly units, elutable assemblies suitable for adsorbing a biomolecule, kits or storage units. In one embodiment, a storage apparatus is capable of maintaining the absorbed assembly unit, elutable assembly suitable for adsorbing a biomolecule, kit or storage unit at a temperature at about −20° C., at about 4° C., at 4-10° C., at 10-20° C., at 20-30° C., at 30-40° C., at 40-50° C., at 50-60° C., at 60-70° C., or at 70-80° C.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

### Example 1

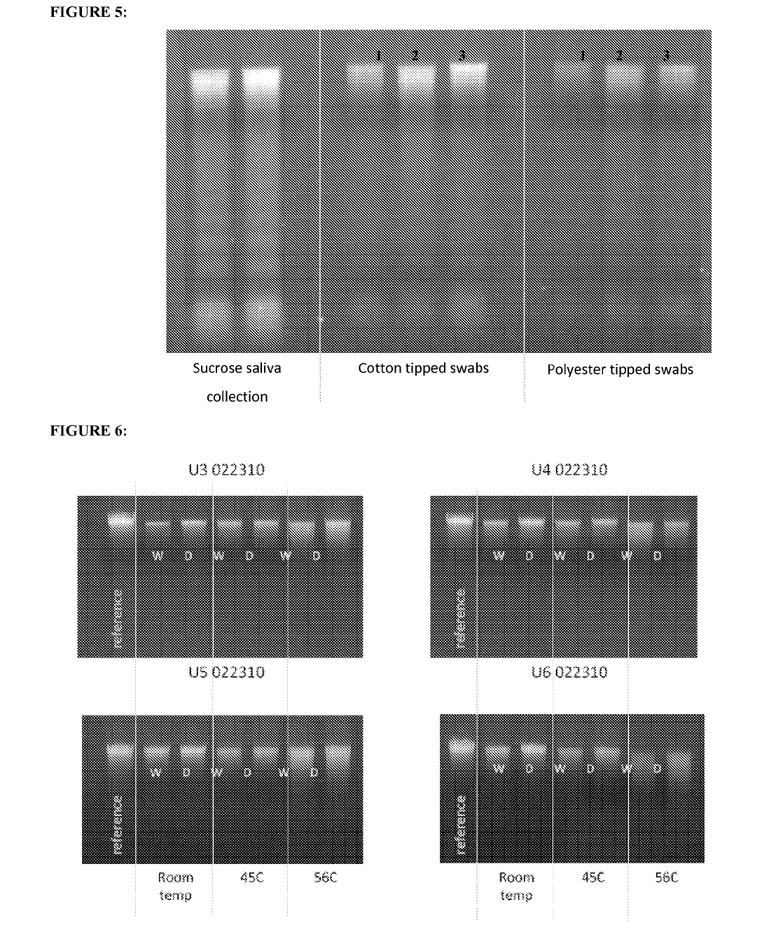

This Example, in FIG. 5, shows results from recovery of saliva samples applied to excess sucrose and air dried overnight at ambient temperature, in accordance with an embodiment of the invention. Following rehydration in water, cells are spun down for subsequent DNA recovery using a standard Qiagen protocol. The resulting DNA is run on an agarose gel and stained with ethidium bromide for visualization. Buccal samples collected using cotton swabs (B) or polyester swabs (C) are allowed to, air dry after collection (1), dipped in sucrose solution (2), or in sucrose crystals (3). DNA is recovered using standard qiagen protocol and run on an agarose gel.

### Example 2

This Example, in FIG. 6, shows recovery results from whole blood storage on an assembly of Sucrose using the following protocol: 200 ul each of 4 different blood lots were applied to 1.2 g of sucrose matrix. Some samples were immediately sealed (indicated by a “W”) or air-dried for 48 hours at room temperature (indicated by a “D”) prior to sealing. Samples were stored in the crystalline sucrose assembly at the indicated temp for 30 days before recovery via rehydration, then DNA purification via Qiagen Mini-column technology. The resulting DNA was then analyzed by agarose electrophoresis, under conditions where DNA>40 Kb will appear as a single collapsed band. A Reference blood sample was frozen at −20 c and similarly purified.

### Example 3

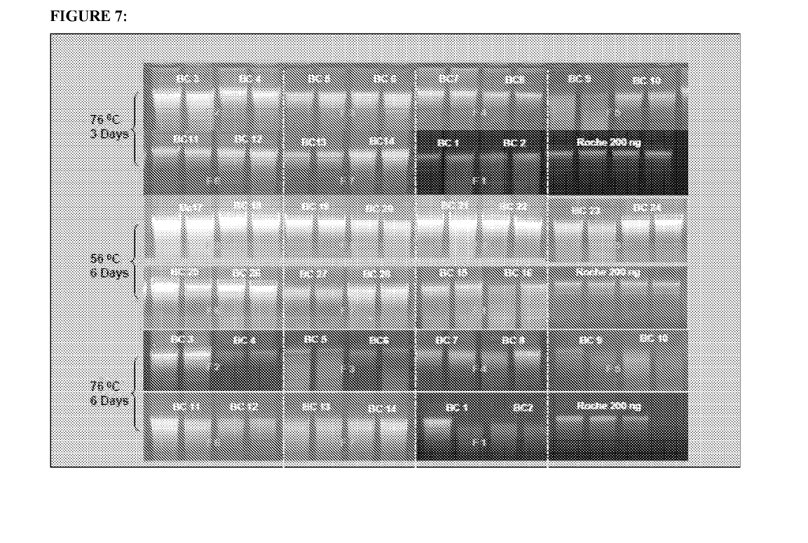

This example, in FIG. 7, shows results from buffy coat storage on the assembly of Sucrose, using the following protocol: Blood from different healthy donors was fractionated by centrifugation to yield an enriched buffy coat fraction, 30 uL of which was then applied to 0.2 g of sucrose matrix amended with a number of formulations. F1 (H2O), F2 (Lysine), F3 (Lysine, KCl, potassium sorbate, pyruvate, ATA), F4 (Lysine, KCl, potassium sorbate, pyruvate, ATA, twice the concentration of F3), F5 (Lysine, potassium sorbate, pyruvate, ATA), F6 (Lysine, potassium sorbate, pyruvate, ATA—twice the concentration of F5), and F7 (Lysine, potassium sorbate, pyruvate, ATA, histidine). Samples were air-dried and then stored at room temperature (RT), 56 C or 76 C for up to 6 days. This served to screen alternative Crystal Matrix surface enhancements. DNA was recovered by solubilizing the buffy coat sugar complex in PBS followed by Qiagen mini-column technology.

What is claimed is:

1. A method for stabilizing a biological sample comprising contacting said biological sample with an assembly of particles,
wherein said particles:
(1) have a long dimension no greater than 5 mm,
(2) comprise an insoluble core, and
(3) have an outer surface having a contact angle of wetting for water less than 50 degrees;
wherein said assembly has an interstitial space of greater than 10% of the total volume of the assembly;
thereby capturing free liquid molecules from said sample.

2. The method of claim 1, wherein said assembly comprises a stabilizer.

3. The method of claim 1, wherein the assembly comprises spherical particles and the spherical particles are contacted with a sample comprising fluid having a volume that is up to 80% of the total volume of the spherical particles in the assembly.

4. The method of claim 1, further comprising air-drying the sample and assembly of particles after said contacting step.

5. The method of claim 4, further comprising rehydrating said sample by applying a controlled volume of a liquid hydrant to said assembly of particles thereby recovering at least a portion of said sample.

6. The method of claim 5, wherein said controlled volume of said liquid hydrant is less than two times the volume of said assembly of particles.

7. The method of claim 1, wherein said assembly has a size range from 10-20 mm³, 20-30 mm³, 30-50 mm³, 50-100 mm³, 100-200 mm³, 200-500 mm³, 500-1000 mm³, 1-5 cm³ or 5-10 cm³.

8. The method of claim 1, wherein said assembly forms an aggregate.

9. The method of claim 1, wherein said particles have a long dimension in the range from 0.1 mm and 1 mm.

10. The method of claim 1, wherein the insoluble core comprises a plastic material.

11. The method of claim 10, wherein a plastic material is selected from polycarbonate, polyurethane, polyalkelene glycol, polypropylene, nylon, and polyethylene.

12. The method of claim 1, wherein said particles have an outer surface contact angle is less than 25°.

13. The method of claim 1, wherein said assembly has an interstitial space of greater than 30% of the total volume of the particles in the assembly.

14. The method of claim 1, wherein said assembly has an interstitial space of greater than 50% of the total volume of the particles in the assembly.

15. The method of claim 1, wherein said biological sample comprises blood, urine, saliva, sputum, nasal discharges, a lavage, serum, plasma, mucus, cerebrospinal fluid, stool or semen.

16. The method of claim 1, wherein said biological sample is carried on a solid support.

17. The method of claim 2, wherein the stabilizer comprises an anti-oxidant, a nuclease inhibitor or a protease inhibitor.

18. The method of claim 1, wherein said particles comprise a surface layer that is water soluble.

19. The method of claim 1, wherein said contacting step results in solvation of a surface layer of said particles.

20. The method of claim 1, wherein the particles are contacted with a sample comprising fluid having a volume that is up to 50% of the volume of the particles in the assembly.
